(19) United States
(12) Patent Application Publication
(10) Pub. No.: US 2009/0117069 A1
(43) Pub. Date: May 7, 2009

(54) SUBLIMABLE COMPOSITION FOR CONTROLLING ODOR

(75) Inventor: ERIC L. MAINZ, Goddard, KS (US)

Correspondence Address: INVISTA NORTH AMERICA S.A.R.L., THREE LITTLE FALLS CENTRE/1052, 2801 CENTERVILLE ROAD, WILMINGTON, DE, 19808, US

(73) Assignee: INVISTA NORTH AMERICA S.A R.L., Wilmington, DE (US)

(21) Appl. No.: 12/259,486

(22) Filed: Oct. 28, 2008

Related U.S. Application Data
(60) Provisional application No. 60/984,889, filed on Nov. 2, 2007.

(51) Int. Cl. A61L 9/04

(52) U.S. Cl. 424/76.4; 424/76.2

(57) ABSTRACT

The invention relates to a sublimable composition comprising cyclododecane and adipic acid and an optional diluent promoting sublimation of the composition. The sublimable composition is provided with a fragrance which is carried into the air by the sublimable composition to ameliorate or otherwise mask objectionable odors, particularly those odors from toilets, urinals and the like.

### CROSS-REFERENCE TO RELATED APPLICATION

This application claims benefit of priority from U.S. Provisional Application No. 60/984,889, filed Nov. 2, 2007. This application hereby incorporates by reference U.S. Provisional Application No. 60/984,889 in its entirety.

### FIELD OF THE INVENTION

The invention relates to a sublimable composition for controlling odors having improved properties. More particularly the invention relates to a composition for use in toilets, urinals and places where objectionable odors can be ameliorated using a sublimable composition capable of carrying a fragrance or odor masking agent.

### BACKGROUND OF THE INVENTION

It is known to use para-dichlorobenzene (CAS Reg. No. 106-46-7), camphor (CAS Reg. No 76-22-2 for synthetic DL-camphor) and other sublimable organic compounds in control of odors in toilets and urinals. Such materials are often known as urinal cakes. Generally formed into right cylindrical shape these cakes are provided in a urinal directly or associated with a receiver structure, the structure usually made of plastic. These compounds used in urinal cakes may serve as carriers of fragrance with the intent of counteracting objectionable odors. The para-dichlorobenzene, camphor and some other organic compounds have significant vapor pressure (and are sublimable) when in the solid form at room temperature. The state of California has banned the use of para-dichlorobenzene as a room and urinal deodorant due to its high vapor pressure and VOC (volatile organic compound) status. An alternative composition is desirable, having both a low solubility in water and/or urine, capable of carrying a fragrance and sublimable. Such sublimable compositions carry and slowly release a fragrance and/or deodorant combined with the composition.

### SUMMARY OF THE INVENTION

The Applicants have discovered a composition, a novel formulation, of cyclododecane (CAS Reg. No. 294 62 2) and adipic acid (CAS Reg. No.124 04 9) optionally combined with inorganic or organic soluble salts and a fragrance and/or deodorant ingredients. Disclosed herein is a sublimable composition useful in the control of odors. The disclosed sublimable composition may also be useful as a fragrance carrier in the treatment and masking of odors.

The sublimable composition for use in odor control formulations, air freshening or deodorizing may comprise cyclododecane in an amount from about 0.1 to about 99.9 percent by weight and adipic acid in an amount from about 99.9 to 0.1 percent by weight.

The sublimable composition for use in odor control formulations, air freshening or deodorizing comprising cyclododecane and adipic acid may further comprise a sublimation assist component and diluent component. The sublimable composition for use in odor control formulations, air freshening or deodorizing comprising cyclododecane and adipic acid and a sublimation assist component and diluent component comprising sodium sulfate and/or sodium borate. The sublimable composition for use in odor control formulations, air freshening or deodorizing comprising cyclododecane and adipic acid may further comprise components selected from the group consisting of: fragrances; air fresheners; oxidants; monobasic carboxylic acids; dibasic carboxylic acids; the potassium, lithium, and magnesium salts of monobasic carboxylic acids; the potassium, lithium, and magnesium salts of dibasic carboxylic acids; amino acids.

The sublimable composition for use in odor control formulations, air freshening or deodorizing may comprise cyclododecane in an amount from about 10 to 80 percent by weight, adipic acid in an amount from about 3 to 80 percent by weight, sodium sulfate in an amount from about 1 to 90 percent by weight and fragrance in an amount from about 0.1 to 10 percent by weight.

Embodiments may show an improved time release of a deodorant carried by the novel formulation disclosed herein. This formulation can be easily formed into powders, granules, granulate, flakes, pellets and solid blocks of practically any size and geometric shape.

### DETAILED DESCRIPTION

In general, odors which may result from organic wastes, human and animal body fluids, molds and mildew, and decaying plant and animal matter are detectable by human olfactory perception. Such detectable odors are found to be objectionable in the home, in public spaces and in work spaces. The sublimable compositions disclosed herein are intended for control of such odors.

These sublimable compositions may be used to treat odors detectable from bathrooms, restrooms, chemical toilets, urinals, waste holding tanks, closet spaces or living rooms, and any closed or open air space where odors may be present continuously or periodically.

The sublimable composition for use in odor control formulations, air freshening or deodorizing may comprise a continuously varying composition of cyclododecane in an amount from about 0.1 to about 99.9 percent by weight with adipic acid in an amount from about 99.9 to 0.1 percent by weight.

The sublimable composition for use in odor control formulations, air freshening or deodorizing comprising cyclododecane and adipic acid may further comprise a sublimation assist component and diluent component comprising either sodium sulfate or sodium borate or both.

The sublimable composition for use in odor control formulations, air freshening or deodorizing comprising cyclododecane and adipic acid may further comprise components selected from the group consisting of: fragrances; air fresheners; oxidants; monobasic carboxylic acids; dibasic carboxylic acids; the potassium, lithium, and magnesium salts of monobasic carboxylic acids; the potassium, lithium, and magnesium salts of dibasic carboxylic acids; amino acids.

The sublimable composition for use in odor control formulations, air freshening or deodorizing may comprise cyclododecane in an amount from about 10 to 80 percent by weight, adipic acid in an amount from about 3 to 80 percent by weight, sodium sulfate in an amount from about 1 to 90 percent by weight and fragrance in an amount from about 0.1 to 10 percent by weight.

Results of the Applicants' testing of such a composition show an improved time release of a deodorant carried by the novel formulation disclosed herein. This formulation can be easily formed into powders, granules, granulate, flakes, pellets and solid blocks of practically any size and geometric shape.

Odor control compositions useful in the practice of the Applicants' disclosures are solid materials at ambient temperature. The formulations are based on a combination of cyclododecane, plus monobasic and/or dibasic organic acids. Illustrative acids, include, among others, citric acid, formic acid, acetic acid, propionic, butyric, oxalic, phthalic, N-acetylglycine, acetylsalicylic acid, fumaric acid, glycolic acid, iminodiacetic acid, itaconic acid, laric acid, lactic acid, maleic acid, malic acid, nicotinic acid, 2-pyrrolidone-5-carboylic acid, salicylic acid, succinamic acid, succinic acid, ascorbic acid, aspartic acid, glutamic acid, glutaric acid, malonic acid, pyruvic acid, sulfonyldiacetic acid, benzoic acid, epoxysuccinic acid, adipic acid, thiodiacetic acid and thioglycolic acid. Other acids that can be employed are the mono- and dicarboxylic acids composed of carbon, hydrogen, oxygen and nitrogen (also called herein after amino acids). These acids can be used in combination with other components such as inorganic materials like salts of chloride, sulfate, borates, nitrates, etc or any salt having some solubility in water and other fluids such as urine. These salts can be in the form of any of the alkali metals (sodium, lithium, potassium, etc.) or alkaline earth metals (magnesium, calcium, etc.) or the transition metal salts having some water solubility). It is also possible to include hydroxyl compounds to produce organic acid salts in situ in the end use application (such as a urinal deodorant block) or to mix and react the hydroxyl compounds with the mono or dibasic acids prior to forming into the end use solid deodorant product. A typical end use product form would be formed as a block or disc of varying sizes. Size and shape would be dictated by the end use and the available equipment for forming the product. The formulated product could also take the form of powder, granules, flakes, prills, balls, shaped blocks, etc. Fragrance can be added to the solid forms of the formulations at any stage of the formulation process, prior to or after the solid has been shaped into its final form.

The selection of the aroma chemicals forming the added fragrance or air freshener composition should be such that the fragrance composition does not unduly evaporate or have such a high volatility that it does not provide a lasting effect to ameliorate objectionable odor. Advantageously, the fragrance constituent will contain mixed portions of high boiling point (low vapor pressure) aroma chemicals and lower boiling point (higher vapor pressure) aroma chemicals that are useful for providing a fragrance.

Perfumers skilled in the art will formulate fragrances such that the composition will have a desirable odor. For example, 50% wt. of the fragrance constituent may consists of aroma chemicals having a vapor pressure of less than 0.1 mm Hg at 25° C. and exhibit a human perception odor threshold of less than 5 nanograms per liter.

Examples of volatile, low boiling, fragrence ingredients are: anethole, benzaldehyde, benzyl acetate, benzyl alcohol, benzyl formate, iso-bomyl acetate, camphene, cis-citral (neral), citronellal, citronellol, citronellyl acetate, para-cymene, decanal, dihydrolinalool, dihydromyrcenol, dimethyl phenyl carbinol, eucalyptol, geranial, geraniol, geranyl acetate, geranyl nitrile, cis-3-hexenyl acetate, hydroxycitronellal, d-limonene, linalool, linalool oxide, linalyl acetate, linalyl propionate, methyl anthranilate, alpha-methyl ionone, methyl nonyl acetaldehyde, methyl phenyl carbinyl acetate, laevo-menthyl acetate, menthone, iso-menthone, myrcene, myrcenyl acetate, myrcenol, nerol, neryl acetate, nonyl acetate, phenyl ethyl alcohol, alpha-pinene, beta-pinene, gamma-terpinene, alpha-terpineol, beta-terpineol, terpinyl acetate, and vertenex (para-tertiary-butyl cyclohexyl acetate). Some natural oils also contain large percentages of highly volatile perfume ingredients. For example, lavandin contains as major components: linalool; linalyl acetate; geraniol; and citronellol. Lemon oil and orange terpenes both contain about 95% of d-limonene.

Examples of less volatile, high boiling perfume ingredients are: amyl cinnamic aldehyde, iso-amyl salicylate, benzophenone, benzyl salicylate, beta-caryophyllene, cedrene, cinnamic alcohol, coumarin, dimethyl benzyl carbinyl acetate, ethyl vanillin, ethylene brassylate, eugenol, iso-eugenol, flor acetate, galaxolide (1,3,4,6,7,8-hexahydro-4,6,6,7,8,8-hexamethyl-cyclopenta-gama-2-b-enzopyran), heliotropine, 3-cis-hexenyl salicylate, hexyl cinnamic aldehyde, hexyl salicylate, lilial (para-tertiarybutyl-alpha-methyl hydrocinnamic aldehyde), lyral (4-(4-hydroxy-4-methyl pentyl)-3-cyclohexene-10-carboxaldehyde), methyl cedrylone, methyl dihydro jasmonate, gamma-methyl ionone, methyl-beta-naphthyl ketone, musk indanone, musk ketone, musk tibetene, nerolidol, patchouli alcohol, phenylethyl phenyl acetate, phenyl hexanol, beta-selinene, trichloromethyl phenyl carbinyl acetate, triethyl citrate, vanillin, and veratraldehyde. Cedarwood terpenes are composed mainly of alpha-cedrene, beta-cedrene, and other C15H24 sesquiterpenes.

The fragrance constituent may be present in any effective amount. Advantageously the fragrance constituent comprises not more than 20% wt. of the effective composition delivery system. The delivery system comprises the sublimable composition, optional sublimation assist additives and diluents.

### Test Methods

The performance of the compositions (or formulations) described in the Examples below were evaluated using this procedure. Each solid block (or cake) were exposed to the atmosphere in a laboratory fume exhaust hood with air flowing constantly over the blocks. The blocks were periodically subjected to being submerged in water held at a temperature of 100° degrees Fahrenheit (38° C.). The blocks were submerged for a soak time of five minutes. Upon removal from the water soak, the blocks were patted dry with paper towel and weighed to record weight lost. The blocks were then placed back into the exhaust hood and air flow. The water submergence, soak and weighing procedure was typically repeated twice per day, five days per week. Weight loss of the test blocks was recorded and compared in order to track block performance in terms of block weight loss. Normally weight loss correlated with fragrance release. The shape and integrity of the blocks were also observed and recorded for a period of 8 to 14 days, depending on the specific block.

### EXAMPLES

Examples of compositions formulated for odor control are described by the following examples.

### Example 1

Formulation “A” was prepared from: cyclododecane (CDD) (available from INVISTA® Specialty Intermediates C12; www.intermediates.invista.com), INVISTA-RGA Grade adipic acid (AA) (available from INVISTA® Intermediates; www.intermediates.invista.com), and puffed sodium borate (PSB) from Rio Tinto BORAX.

The formulation consisted of: 567 grams of CDD, 250 grams of AA and 123 grams of PSB. The formulation composition was:

CDD60.3 wt percent

AA 26.6 wt. percent

PSB 13.1 wt. percent.

Deodorant blocks containing about 2 ounces of the above formulation were formed using the following procedure. The CDD was heated in a steam bath until melted. The AA was added to the plastic bottle containing the CDD melt. The components were mixed by shaking vigorously. Next the PSB was added and mixed by shaking. The mixture was reheated on the steam bath to produce a pourable slurry melt. The melt was poured onto metal baking sheets into a layer approximately one-eight inch in thickness and allowed to cool. The cooled mass was broken into large flake pieces and placed in a heavy plastic bag. The large pieces were broken by striking the bag containing the cooled melt flakes with a leather mallet.

The crushed material was formed into solid blocks by pressing in a mold using a hydraulic press. A mold was prepared consisting of a piece of aluminum tubing cut to a length of about 4 inches and having an inside diameter of 2.07 inches. A plunger rod fitting loosely into the pipe was cut from a solid polymer rod. The plunger rod had dimensions of 2.00 inches diameter and a length of 5 inches. A Carver Lab Press, Model C was used to press the crushed melt into blocks with dimension of about 2 inches in diameter by 1 inch in height.

To form the blocks, the tubing mold was placed on an approximate 4 inch by 4 inch piece of stainless steel plate. About 65 grams of the crushed formulation was placed into the mold. The plunger rod was placed in the mold and the assembly was placed on the hydraulic press ram. The press was activated by pumping a hydraulic jack that was part of the press. Pressure was applied by pumping the jack handle until a pressure gauge on the hydraulic system indicated a pressure of 5000 PSIG. As the formulation particles were pressed together, the pressure would decline and more pressure was applied. Pressure was reapplied until the pressure drop rate declined significantly. Next the pressure was released and the stainless steel plate was removed from the assembly. A slightly larger diameter piece of aluminum tubing was placed under the aluminum tubing mold. Pressure was reapplied to the mold plunger rod to press the deodorant block from the mold.

It is also possible to form blocks of this formulation (and others of this invention) by casting the formulation melt into molds of various configurations.

### Example 2

Formulation “B” was prepared composed of CDD, RGA Grade AA and sodium sulfate (SS) Detergent Grade, from Saskatchewan Minerals.

The formulation consisted of: 489.7 grams of CDD, 245.8 grams of AA and 244.2 grams of SS. The formulation composition was:

CDD50.0 wt percent

AA 25.1 wt. percent

SS 24.9 wt. percent.

Deodorant blocks were formed using a procedure similar to that described in Example 1. Once the three components had been mixed thoroughly by placing in a plastic bottle and shaking, it was noted that upon standing, some ingredient began to settle to the bottom of the bottle. The mixture was allowed to cool until the viscosity increased enough to help suspend the undissolved solids in the formulation melt. The melt was poured onto metal baking sheets into a layer approximately one-eight inch in thickness and allowed to cool. The procedure described in Example 1 was followed to form the formulation into approximately 2 ounce blocks.

### Example 3

Formulation “C” consisted of a formulation “A” block with fragrance added. Fragrance was added to a block prepared as described in Example 1. A block weighing 64.6 grams was placed in 750 milliliter reaction kettle. The kettle was fitted with a septum port. Vacuum was applied to the kettle for a time period of 10 minutes at a pressure of 15 mm Hg. 2.35 grams of lavender fragrance oil (CAS registry number 8000-28-0) solution was added to the kettle via a syringe needle placed through the septum port on the kettle. It was observed that the deodorant block adsorbed the fragrance oil. The fragrance oil loading was about 3.5 weight percent.

### Example 4

Formulation “D” consisted of a formulation “B” block with fragrance added. Fragrance was added to a block prepared as described in Example 3. A block weighing 63.7 grams was placed in 750 milliliter reaction kettle. The kettle was fitted with a septum port. Vacuum was applied to the kettle for a time period of 10 minutes at a pressure of 15 mm Hg. 1.97 grams of lavender fragrance oil solution was added to the kettle via a syringe needle placed through the septum port on the kettle. It was observed that the deodorant block adsorbed the fragrance oil. The fragrance oil loading was about 3 weight percent.

Methods for adding fragrance or other additives to the blocks may include but are not limited to the addition of the fragrance or deodorant additives to any or all of the formulation components prior to mixing together and prior to melting the CDD. The additives can include liquids and solids. Fragrances are available as liquids and also loaded onto solid carriers. Many forms of fragrance are available and known to the art and especially to a skilled person in perfumery. The addition of solid fragrance carriers and solid fragrances and solid deodorant additives can be accomplished as described above as well as by other methods know to the art. Solid containing fragrance(s) and solid deodorant additives can be in the form of powder, flakes, granules, pellets etc.

### Block Performance

The performance of the formulations described in Examples 1, 2, 3 and 4 were evaluated using the test method described in the foregoing section.

Weight loss performance of the four test blocks evaluated is shown in the table below.

| | | | Test | Avg. wt. |
|---|---|---|---|---|
| | Formulation | | Duration | loss/day |
| Sample (No.) | Example | Fragrance | Days | Grams |
| A (1212) | 1 | no | 14 | 1.6 |
| C (1201) | 2 | yes | 14 | 1.5 |
| B (1415) | 3 | no | 11 | 2.6 |
| D (1403) | 4 | yes | 8 | 2.3 |

Formulation A and C, both containing CDD, AA and PSB had a weight loss rate of 1.5 to 1.6 grams per day. Based on a starting weight of about 63 grams, formulation C would be expected to sublime, dissolve and release fragrance for a period of about 40 days.

Formulation B and D, both containing CDD, AA and SS had a weight lost rate of 2.3 to 2.6 grams per day. Based on a starting weight of about 63 grams, formulation D would be expected to sublime, dissolve and release fragrance for a period of about 27 days.

The formulation can be easily adjusted to modify the sublimation and dissolution rate by adjusting the ratios of the CDD, AA and other additives.

The foregoing disclosure constitutes a description of specific embodiments illustrating how the invention may be used and applied. Such embodiments are only exemplary. The invention in its broadest aspects is further defined in the claims which follow. These claims and terms used therein are to be taken as variants of the invention described. These claims are not restricted to such variants but are to be read as covering the full scope of the invention implicit within the disclosure herein.

What is claimed:

1. A sublimable composition comprising cyclododecane and adipic acid.

2. The sublimable composition of claim 1 comprising cyclododecane in an amount from about 0.1 to about 99.9 percent by weight and adipic acid in an amount from about 99.9 to 0.1 percent by weight.

3. The sublimable composition of claim 1 further comprising a diluent promoting sublimation of the composition.

4. The sublimable composition of claim 3 wherein said diluent comprises sodium sulfate or sodium borate or both.

5. The sublimable composition of claim 1 further comprising components selected from the group consisting of: fragrances; air fresheners; oxidants; amino acids; monobasic carboxylic acids; dibasic carboxylic acids; the potassium, lithium, and magnesium salts of monobasic carboxylic acids; the potassium, lithium, and magnesium salts of dibasic carboxylic acids.

6. A solid cake for use in odor control formulations, air freshening or deodorizing comprising cyclododecane in an amount from about 10 to 80 percent by weight, adipic acid in an amount from about 3 to 80 percent by weight, sodium sulfate in an amount from about 1 to 90 percent by weight and fragrance in an amount from about 0.1 to 10 percent by weight.
